(19) United States
(12) Patent Application Publication
(10) Pub. No.: US 2025/0135062 A1
(43) Pub. Date: May 1, 2025

(54) ODOUR COUNTERACTION

(71) Applicant: GIVAUDAN SA, VERNIER (CH)

(72) Inventor: Lin Mei HE, Shanghai (CN)

(21) Appl. No.: 18/687,741

(22) PCT Filed: Aug. 11, 2022
(86) PCT No.: PCT/EP2022/072550

§ 371 Date: Feb. 28, 2024

(30) Foreign Application Priority Data
Aug. 31, 2021 (WO) PCT/CN2021/115511

(51) Int. Cl. A61L 15/22; A61L 15/46; A61L 101/48; A61L 101/50

(52) U.S. Cl. CPC A61L 15/225; A61L 15/46; A61L 2101/48; A61L 2101/50

(57) ABSTRACT

A method of providing fragrance to a product releasable on exposure of the product to humidity, comprising
(a) the encapsulation of fragrance in a particulate matrix by dispersing the fragrance in a solution of the matrix material and spray-drying the dispersion to give fragrance-containing particles;
(b) dispersing the particles in an organic liquid non-solvent for the matrix material; and
(c) adding the resulting particles to the product.

The particles thus treated do not separate so readily from the product, resulting in a longer-lasting fragrance.

This disclosure relates to the provision of fragrance in products that are exposed to humidity.

It is desirable to have fragrance in certain products that are exposed to humidity, especially when such exposure can result in undesirable odours, Examples of such products include animal litters, sanitary products, diapers, air fresheners, humidity absorbers and air-conditioning systems. The fragrance must naturally be in such a form that it is released at the appropriate time, in order to combat an undesirable odour. This naturally precludes fragrance being simply added to the product.

One method used has been encapsulation of the fragrance, such that it is released on exposure to moisture. There are a number of methods of doing this, but one particularly useful one, from both cost and effectiveness points of view has been the encapsulation of the fragrance in starch. To achieve this, the fragrance is dispersed in a solution of a water-soluble polymer (usually, but not always, starch), and the dispersion is then spray-dried to produce fragrance-containing granules.

While this method of fragrance incorporation works in many applications, it is less useful in some others. For example, if the granules are added to an animal litter, such as a cat litter, their fine size means that they tend to gravitate to the bottom of any litter tray, thus reducing their effectiveness.

It has now been found that it is possible to overcome this problem and to use starch encapsulates in circumstances where they would normally be less effective. There is therefore provided a method of providing fragrance to a product releasable on exposure of the product to humidity, comprising
(a) the encapsulation of fragrance in a particulate matrix by dispersing the fragrance in a solution of the matrix material and spray-drying the dispersion to give fragrance-containing particles;
(b) dispersing the particles in an organic liquid non-solvent for the matrix material; and
(c) adding the resulting particles to the product.

The fragrance may be any desirable fragrance that is one or more of known fragrance ingredients. Fragrance ingredients that may be used include, but are not limited to ADOXAL™ (2,6,10-trimethylundec-9-enal); AGRUMEX™ (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE ISO C 11 ((E)-undec-9-enal); ALDEHYDE MANDARINE 10%/TEC ((E)-dodec-2-enal); ALLYL AMYL GLYCOLATE (allyl 2-(isopentyloxy)acetate); ALLYL CYCLOHEXYL PROPIONATE (allyl 3-cyclohexylpropanoate); ALLYL OENANTHATE (allyl heptanoate); AMBER CORE™ (1-((2-(tert-butyl)cyclohexyl)oxy)butan-2-ol); AMBERMAX™ (1,3,4,5,6,7-hexahydro-beta, 1, 1,5,5-pentamethyl-2H -2,4a-methanonaphthal-ene-8-ethanol); AMYL SALICYLATE (pentyl 2-hydroxybenzoate); APHERMATE (1-(3,3-dimethylcyclohexyl)ethyl formate); BELAMBRE™ ((1R,2S,4R)-2′-isopropyl-1,7,7-trimethylspiro[bicyclo[2.2.1]heptane-2,4′-[1,3]dioxane]); BIGARYL (8-(sec-butyl)-5,6,7,8-tetrahydroquinoline); BOISAMBRENE FORTE™ ((ethoxymethoxy)cyclododecane); BOISIRIS™ ((1S,2R,5R)-2-ethoxy-2,6,6-trimethyl-9-methylenebicyclo[3.3.1]nonane); BORNYL ACETATE ((2S,4S)-1,7,7-trimethylbicyclo[2.2.1]heptan-2-yl acetate); BUTYL BUTYRO LACTATE (1-butoxy-1-oxopropan-2-yl butyrate); BUTYL CYCLOHEXYL ACETATE PARA (4-(tert-butyl)cyclohexyl acetate); CARYOPHYLLENE ((Z)-4, 11,11-trimethyl-8-methylenebicyclo[7.2.0]undec-4-ene); CASHMERAN™ (1,1,2,3,3-pentamethyl-2,3,6,7-tetrahydro-1H-inden-4(5H)-one); CASSYRANE™ (5-tert-butyl-2-methyl-5-propyl-2H-furan); CITRAL ((E)-3,7-dimethylocta-2,6-dienal); CITRAL LEMAROME™ N ((E)-3,7-dimethylocta-2,6-dienal); CITRATHAL™ R ((Z)-1, 1-diethoxy-3,7-dimethylocta-2,6-diene); CITRONELLAL (3,7-dimethyloct-6-enal); CITRONELLOL (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CITRONELLYL PROPIONATE (3,7-dimethyloct-6-en-1-yl propionate); CLONAL (dodecanenitrile); CORANOL (4-cyclohexyl-2-methylbutan-2-ol); COSMONE™ ((Z)-3-methylcyclotetradec-5-enone); CYCLAMEN ALDEHYDE (3-(4-isopropylphenyl)-2-methylpropanal); CYCLOGALBANATE (allyl 2-(cyclohexyloxy)acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA ((E)-1-(2,6,6-trimethylcyclohex-3-en-1-yl)but-2-en-1-one); DECENAL-4-TRANS ((E)-dec-4-enal); DELPHONE (2-pentylcyclopentanone); DIHYDRO ANETHOLE (propanedioic acid 1-(1-(3,3-dimethylcyclohexyl)ethyl)3-ethyl ester); DIHYDRO JASMONE (3-methyl-2-pentylcyclopent-2-enone); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butyrate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DUPICAL™ ((E)-4-((3aS,7aS)-hexahydro-1H-4,7-methanoinden-5(6H)-ylidene)butanal); EBANOL™ ((E)-3-methyl-5-(2,2,3-trimethylcyclopent-3-en-1-yl)pent-4-en-2-ol); ETHYL CAPROATE (ethyl hexanoate); ETHYL CAPRYLATE (ethyl octanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL OENANTHATE (ethyl heptanoate); ETHYL SAFRANATE (ethyl 2,6,6-trimethylcyclohexa-1,3-diene-1-carboxylate); EUCALYPTOL ((1s,4s)-1,3,3-trimethyl-2-oxabicyclo[2.2.2]octane); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FIXOLIDE™ (1-(3,5,5,6,8,8-hexamethyl-5,6,7,8-tetrahydronaphthalen-2-yl)ethanone); FLORALOZONE™ (3-(4-ethylphenyl)-2,2-dimethylpropanal); FLORHYDRAL (3-(3-isopropylphenyl)butanal); FLOROCYCLENE™ ((3aR,6S,7aS)-3a,4,5,6,7,7a-hexahydro-1H-4,7-methanoinden-6-yl propionate); FLOROPAL™ (2,4,6-trimethyl-4-phenyl-1,3-dioxane); FRESKOMENTHE™ (2-(sec-butyl)cyclohexanone); FRUITATE ((3aS,4S,7R,7aS)-ethyl octahydro-1H-4,7-methanoindene-3a-carboxylate); FRUTONILE (2-methyldecanenitrile); GALBANONE™ PURE (1-(3,3-dimethylcyclohex-1-en-1-yl)pent-4-en-1-one); GARDOCYCLENE™ ((3aR,6S,7aS)-3a,4,5,6,7,7a-hexahydro-1H-4,7-methanoinden-6-yl isobutyrate); GERANIOL ((E)-3,7-dimethylocta-2,6-dien-1-ol); GERANYL ACETATE SYNTHETIC ((E)-3,7-dimethylocta-2,6-dien-1-yl acetate); GERANYL ISOBUTYRATE ((E)-3,7-dimethylocta-2,6-dien-1-yl isobutyrate); GIVESCONE™ (ethyl 2-ethyl-6,6-dimethylcyclohex-2-enecarboxylate); HABANOLIDE™ ((E)-oxacyclohexadec-12-en-2-one); HEDIONE™ (methyl 3-oxo-2-pentylcyclopentaneacetate); HERBANATE™ ((2S)-ethyl 3-isopropylbicyclo[2.2.1]hept-5-ene-2-carboxylate); HEXENYL-3-CIS BUTYRATE ((Z)-hex-3-en-1-yl butyrate); HEXYL CINNAMIC ALDEHYDE ((E)-2-benzylideneoctanal); HEXYL ISOBUTYRATE (hexyl isobutyrate); HEXYL SALICYLATE (hexyl 2-hydroxybenzoate); INDOFLOR™ (4,4a,5,9b-tetrahydroindeno[1,2-d][1,3]dioxine); IONONE BETA ((E)-4-(2,6,6-trimethylcyclohex-1-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER™ (1-(2,3,8,8-tetramethyl-1,2,3,4,5,6,7,8-octahydronaphthalen-2-yl)ethanone); ISOCYCLOCITRAL (2,4,6-trimethylcyclohex-3-enecarbaldehyde); ISONONYL ACETATE (3,5,5-trimethylhexyl acetate); ISOPROPYL METHYL-2-BUTYRATE (isopropyl 2-methyl butanoate); ISORALDEINE™ 70 ((E)-3-methyl-4-(2,6,6-trimethylcyclohex-2-en-1-yl)but-3-en-2-one); JASMACYCLENE™ ((3aR,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); KARANAL™ (5-(sec-butyl)-2-(2,4-dimethylcyclohex-3-en-1-yl)-5-methyl-1,3-dioxane); KOAVONE ((Z)-3,4,5,6,6-pentamethylhept-3-en-2-one); LEAF ACETAL ((Z)-1-(1-ethoxyethoxy)hex-3-ene); LEMONILE™ ((2E,6Z)-3,7-dimethylnona-2,6-dienenitrile); LIFFAROME™ GIV ((Z)-hex-3-en-1-yl methyl carbonate); LILIAL™ (3-(4-(tert-butyl)phenyl)-2-methylpropanal); LINALOOL (3,7-dimethylocta-1,6-dien-3-ol); LINALYL ACETATE (3,7-dimethylocta-1,6-dien-3-yl acetate); MAHONIAL™ ((4E)-9-hydroxy-5,9-dimethyl-4-decenal); MALTYL ISOBUTYRATE (2-methyl-4-oxo-4H-pyran-3-yl isobutyrate); MANZANATE (ethyl 2-methylpentanoate); MELONAL™ (2,6-dimethylhept-5-enal); MENTHOL (2-isopropyl-5-methylcyclohexanol); MENTHONE (2-isopropyl-5-methylcyclohexanone); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL NONYL KETONE EXTRA (undecan-2-one); METHYL OCTYNE CARBONATE (methyl non-2-ynoate); METHYL PAMPLEMOUSSE (6,6-dimethoxy-2,5,5-trimethylhex-2-ene); MYRALDENE (4-(4-methylpent-3-en-1-yl)cyclohex-3-enecarbaldehyde); NECTARYL (2-(2-(4-methylcyclohex-3-en-1-yl)propyl)cyclopentanone); NEOBERGAMATE™ FORTE (2-methyl-6-methyleneoct-7-en-2-yl acetate); NEOFOLIONE™ ((E)-methyl non-2-enoate); NEROLIDYLE™ ((Z)-3,7,11-trimethyldodeca-1,6, 10-trien-3-yl acetate); NERYL ACETATE HC ((Z)-3,7-dimethylocta-2,6-dien-1-yl acetate); NONADYL (6,8-dimethylnonan-2-ol); NONENAL-6-CIS ((Z)-non-6-enal); NYMPHEAL™ (3-(4-isobutyl-2-methylphenyl)propanal); ORIVONE™ (4-(tert-pentyl)cyclohexanone); PARADISAMIDE™ (2-ethyl-N-methyl-N-(m-tolyl)butanamide); PELARGENE (2-methyl-4-methylene-6-phenyltetrahydro-2H-pyran); PEONILE™ (2-cyclohexylidene-2-phenylacetonitrile); PETALIA™ (2-cyclohexylidene-2-(o-tolyl)acetonitrile); PIVAROSE™ (2,2-dimethyl-2-pheylethyl propanoate); PRECYCLEMONET B (1-methyl-4-(4-methylpent-3-en-1-yl)cyclohex-3-enecarbaldehyde); PYRALONE™ (6-(sec-butyl)quinoline); RADJANOLT SUPER ((E)-2-ethyl-4-(2,2,3-trimethylcyclopent-3-en-1-yl)but-2-en-1-ol); RASPBERRY KETONE (N112) (4-(4-hydroxyphenyl)butan-2-one); RHUBAFURANE™ (2,2,5-trimethyl-5-pentylcyclopentanone); ROSACETOL (2,2,2-trichloro-1-phenylethyl acetate); ROSALVA (dec-9-en-1-ol); ROSYFOLIA ((1-methyl-2-(5-methylhex-4-en-2-yl)cyclopropyl)-methanol); ROSYRANE™ SUPER (4-methylene-2-phenyltetrahydro-2H-pyran); SERENOLIDE (2-(1-(3,3-dimethylcyclohexyl)ethoxy)-2-methylpropyl cyclopropanecarboxylate); SILVIAL™ (3-(4-isobutylphenyl)-2-methylpropanal); SPIROGALBANONE™ (1-(spiro[4.5]dec-6-en-7-yl)pent-4-en-1-one); STEMONE™ ((E)-5-methylheptan-3-one oxime); SUPER MUGUET™ ((E)-6-ethyl-3-methyloct-6-en-1-ol); SYLKOLIDE™ ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINOLENE (1-methyl-4-(propan-2-ylidene)cyclohex-1-ene); TERPINYL ACETATE (2-(4-methylcyclohex-3-en-1-yl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); TRIDECENE-2-NITRILE ((E)-tridec-2-enenitrile); UNDECAVERTOL ((E)-4-methyldec-3-en-5-ol); VELOUTONE™ (2,2,5-trimethyl-5-pentylcyclopentanone); VIRIDINE™ ((2,2-dimethoxyethyl)benzene); ZINARINE™ (2-(2,4-dimethylcyclohexyl)pyridine); and mixtures thereof.

A comprehensive list of perfume ingredients that may be encapsulated in accordance with the present invention can be found in the perfumery literature, for example “Perfume & Flavor Chemicals”, S. Arctander, Allured Publishing, 2000.

The matrix material used for the encapsulation may be any water-soluble or water-dispersible material that may be used to encapsulate fragrance. The technology is well known and typical examples of suitable matrix materials include, but are not limited to:
(1) water-soluble or water-dispersible polymer, especially polysaccharides and modified polysaccharides, such as starch and modified starch or dextrin bearing octenyl succinate moieties;
(2) maltodextrin, sugars, sugar alcohols, mannitol, inulin, and trehalose;
(3) natural or synthetic gums such as alginate esters, gum arabic, gum tragacanth and gum karaya, carrageenan, xanthanes, agar-agar, pectines, and pectic acid;
(4) synthetic water-soluble or partially water-soluble, or-degradable polymers, such as (i) vinyl polymers such as poly(vinyl alcohol), poly(vinyl alcohol-vinyl acetate) copolymers, poly(vinyl alcohol-vinyl acetate-sodium vinyl sulfonate) copolymers; poly(vinyl pyrrolidone) and copolymer derivatives such as poly(vinyl pyrrolidone-vinyl acetate), poly(vinylpyrrolidone-vinyl alcohol), poly(vinylpyrrolidone-styrene); (ii) alkali-soluble polymers, e.g. poly(acrylic acid), poly(maleic acid), poly(alkyl(meth)acrylate-(meth)acrylic acid) copolymer, poly(acrylic acid-maleic acid) copolymer, poly(acrylamide), poly(methacrylic acid), poly((meth)acryl amide), (iii) poly(ethylene oxide) and poly(ethylene oxide-propylene oxide) block copolymers; (iv) mixtures thereof;
(5) Biodegradable (bio) polymers such as (i) polyesters, especially polymers of lactic, glycolic, hydroxybutyric acid derivatives such as poly (D,L-lactide), poly (L-lactide), poly (L-lactide-co-glycolide), poly(D,L-lactide-co-glycolide), poly (D,L-lactide-co-glycolide-D-glucose), poly (hydroxybutyric acid) and further derivatives, copolymers or mixtures thereof; (ii) water soluble or water dispersible forms of poly (3-hydroxy butyrate) derivatives; (iii) lignin derivatives such as lignosulphonates, oxylignins and kraft lignins and further derivatives or mixture thereof;

A particular fragrance-encapsulating matrix material is a mixture of modified starch bearing octenyl succinate moieties and mannitol, admixed in a weight % ratio of from 90:10 to from 10:90, particularly from 80:20 to 50:50.

The process and apparatus used for spray drying is commercially available well-known in the art, and its operation in terms of flow rates, temperatures and collection procedures is also well known. It is well within the skill of the art to select suitable parameters for particle production in every individual case.

The organic liquid in which the spray-dried particles are dispersed may be any such liquid that does not dissolve the matrix material. Any such liquid may be used, its actual nature depending on the nature of the selected matrix material. For example, glycerol dissolves mannitol, and thus should not be used with mannitol. However, it may be suitable for use with other matrix materials. In addition, some of the propylene glycol ethers, such as dipropylene glycol dimethyl ether, have unpleasant odours, which may make their use undesirable in some circumstances. Any such selection of combination of liquid and matrix material is within the skill of the art and may be determined by routine, non-inventive experimentation.

Examples include known perfume solvents such as triethyl citrate, isopropyl myristate and diethyl phthalate. Also useful are propylene glycol, dipropylene glycol, 3-methyl 3-methoxy butanol 3-methyl 3-methoxy butanol, liquid medium-chain triglycerides and synthetic isoparaffins, such as those of the Isopar™ range.

Particular examples of suitable organic liquids are polyethylene glycols (PEGs). Any PEG that is a liquid at 20° C. is suitable. As a general rule, this means that the PEG will have a molecular weight in the range of from about 200-700, that is, from the so-called PEG-4 to PEG-14 (the numbers representing the average number of ethylene oxide units present).

The fragrance-containing particles are dispersed in the liquid by simple stirring. The particles may settle on standing, and this presents no problem, provided that they are stirred to provide a homogeneous dispersion prior to being added to the product.

Addition to the product is achieved simply by pouring or spraying on to the product, optionally with mixing. The method used will depend on the nature of the product to be treated, and the appropriate method will be within the ordinary skill of the art.

The encapsulated fragrance may be augmented by liquid fragrance. Any such fragrance may be used. It may be identical to, or different from, the fragrance in the particles. This may be added to the particle dispersion, or it may be added to the product independently of the dispersion. If it is added to the particle dispersion, this should be done immediately prior to application to the product, with stirring, as the mixture is not storage stable.

The product to be treated may be a product that, when subjected to moisture, may result in undesirable odours. Such odours may be inherent in the nature of the moisture, such as urine and catamenial discharges, or it may be something that arises over time in the moist product, such as humidity absorbers in dehumidifiers and air-conditioning systems. Therefore, the disclosure also provides a product that is subject to malodours as a result of moisture contact, the product comprising fragrance-containing particles of fragrance within a matrix, the matrix having been subsequently treated with an organic liquid non-solvent for the matrix material prior to its addition to the product.

The nature and proportion of particles added to a product will depend entirely on the end-use and the desired effect, and this can be ascertained in every case by non-inventive routine experimentation. In a typical example, a tofu-type cat litter will contain from about 0.001-5%, more particularly from 0.02-1% by weight.

Non-limiting examples of products that can advantageously be treated include animal litters, sanitary products, diapers, air fresheners, humidity absorbers and air-conditioning systems.

The disclosure is further described with reference to the following figure and non-limiting examples, in which all parts are by weight.

### DESCRIPTION OF FIGURES

FIG. 1 is a diagrammatic representation of the results of testing for malodour strength and fragrance strength against urine in a sample of cat litter.

### PREPARATION OF PARTICLES

To 5 parts water were added 1.5 parts mannitol 60 and 3 parts modified starch, and the mixture stirred to form a solution. To this solution was added with stirring 3 parts of fragrance and the mixture stirred at 20000 rpm using IKA Turrax 25 Homogenizer for 30 minutes, in order to provide a particle size of 1 um maximum.

12.5 parts of this emulsion was formed into fragrance-containing solid particles by spray-drying. This was done using a high speed centrifugal spray dryer. The parameters for spraying dryer was inlet temperature 140° C. and exit temperature 50° C. The yield of final powder was about 90% with the particle size ranging from 10 um to 150 um.

### Preparation of Cat Litter

To 98 parts of PEG-600 (a polyethylene glycol with an average molecular weight of 600) was added 2 parts of particles, and the mixture was stirred to obtain a homogenous suspension.

This was sprayed immediately on to 100 parts of cat litter base on a conveyor belt. Spraying was done using a WA-200-152P nozzle with 1.5 mm diameter, compressed air with 330 L/min air flow, and 270 ml/mm spray rate.

### Testing of Cat Litter Against a cat Litter in Which the Particles Were not Combined With PEG-600

500 parts of a cat litter that incorporated 1 part of particles without PEG-600 was added to a transparent container. The sedimentation of white particles can immediately be seen at the bottom of the container. In comparison, the same weight of cat litter with containing the same proportion of particles with PEG-600 showed no sedimentation. 100 parts of water was added to both containers to simulate cats' urination. The resulting odours were assessed by a testing panel. The panel found that the cat litter with particles incorporating PEG-600 was considerably more fragrant than was the cat litter with no PEG-600, and that they lasted longer.

A further test was conducted with a commercially-available synthetic cat urine. To three portions each of 50 g of tofu base cat litter were added
nothing (control)
0.05 g fragrance particles as prepared above, without PEG-600
0.05 g fragrance particles as prepared above, with PEG-600

Into each portion, 800 ul synthetic urine was injected. To each portion was added 20 g deionized water and the portions shaken for 10 minutes. They were then allowed to stand for 2 minutes prior to testing.

The three portions were sescesed by a panel of ton testers for both malodour strength and fragrance strength. The ratings were given as follows:
Malodour strength: 0-5, 0—no malodor, 2.5—moderate, 5—very strong malodor
Fragrance strength: 0-5, 0—no strength, 2.5—moderate, 5—very strong

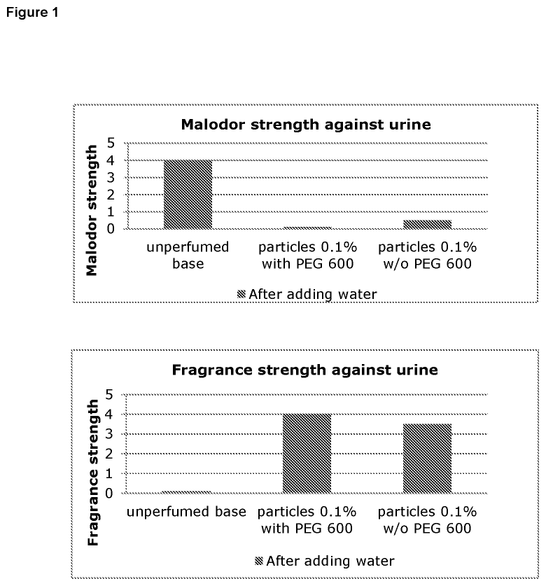

The results are shown in FIG. 1. It can be seen that the particles that incorporate the PEG-600 perform better than the particles without the PEG-600.

1. A method of providing fragrance to a product releasable on exposure of the product to humidity, comprising
(a) the encapsulation of fragrance in a particulate matrix by dispersing the fragrance in a solution of the matrix material and spray-drying the dispersion to give fragrance-containing particles;
(b) dispersing the particles in an organic liquid non-solvent for the matrix material; and
(c) adding the resulting particles to the product.

2. The method according to claim 1, in which the organic liquid non-solvent is selected from triethyl citrate, isopropyl myristate, diethyl phthalate, propylene glycol, dipropylene glycol, polyethylene glycol, 3-methyl 3-methoxy butanol, liquid medium-chain triglycerides, synthetic isoparaffins or combinations thereof.

3. The method according to claim 2, in which the organic liquid non-solvent is selected from polyethylene glycols of molecular weight from about 200 to about 700.

4. The method according to claim 1, in which the matrix material is selected from
(1) water-soluble or water-dispersible polymer;
(2) maltodextrin, sugars, sugar alcohols, mannitol, inulin, or trehalose;
(3) natural or synthetic gums;
(4) synthetic water-soluble or partially water-soluble, or -degradable polymers; and/or
(5) Biodegradable (bio)polymers.

5. The method according to claim 4, in which the matrix material is a mixture of modified starch bearing octenyl succinate moieties and mannitol, admixed in a weight % ratio of from 90:10 to from 10:90.

6. The method according to claim 1, in which the matrix material is a mixture of modified starch bearing octenyl succinate moieties and mannitol, admixed in a weight % ratio of from 90:10 to from 10:90, and the organic liquid non-solvent is a polyethylene glycol (PEGs) having a molecular weight of from about 200 about 700.

7. A product that is subject to malodours as a result of moisture contact, the product comprising fragrance-containing particles of fragrance within a matrix, the matrix having been subsequently treated with an organic liquid non-solvent for the matrix material prior to its addition to the product.

8. The product according to claim 7, in which the product is selected from an animal litter, a sanitary product, a diaper, an air freshener, a humidity absorber and an air-conditioning system.

9. The method according to claim 4, in which the water-soluble or water-dispersible polymer comprises polysaccharides, modified polysaccharides, or combinations thereof.

10. The method according to claim 9, in which the polysaccharides or modified polysaccharides comprise starch, modified starch, dextrin bearing octenyl succinate moieties, or combinations thereof.

11. The method according to claim 4, in which the natural or synthetic gums comprise alginate esters, gum arabic, gum tragacanth and gum karaya, carrageenan, xanthanes, agar-agar, pectines, pectic acid, or combinations thereof.

12. The method according to claim 4, in which the synthetic water-soluble or partially water-soluble, or-degradable polymers comprise (i) vinyl polymers; (ii) alkali-soluble polymers; (iii) poly(ethylene oxide) or poly(ethylene oxide-propylene oxide) block copolymers; or mixtures thereof.

13. The method according to claim 12, in which the (i) vinyl polymers comprise poly(vinyl alcohol), poly(vinyl alcohol-vinyl acetate) copolymers, poly(vinyl alcohol-vinyl acetate-sodium vinyl sulfonate) copolymers, poly(vinyl pyrrolidone) and copolymer derivatives, or mixtures thereof; the (ii) alkali-soluble polymers comprise poly(acrylic acid), poly(maleic acid), poly(alkyl (meth) acrylate-(meth)acrylic acid) copolymer, poly(acrylic acid-maleic acid) copolymer, poly(acrylamide), poly(methacrylic acid), poly((meth)acryl amide), or combinations thereof.

14. The method according to claim 13, in which the poly(vinyl pyrrolidone) or copolymer derivatives comprise poly(vinyl pyrrolidone-vinyl acetate), poly(vinylpyrrolidone-vinyl alcohol), poly(vinylpyrrolidone-styrene), or combinations thereof.

15. The method according to claim 4, in which the biodegradable (bio) polymers comprise (i) polyesters; (ii) water soluble or water dispersible forms of poly (3-hydroxy butyrate) derivatives; (iii) lignin derivatives, or mixture thereof.

16. The method according to claim 15, in which the (i) polyesters comprise polymers of lactic, glycolic, hydroxybutyric acid derivatives, copolymers or mixtures thereof, the (iii) lignin derivatives comprise lignosulphonates, oxylignins and kraft lignins and further derivatives or mixture thereof.

17. The method according to claim 16, in which the polymers of lactic, glycolic, hydroxybutyric acid derivatives comprise poly (D,L-lactide), poly (L-lactide), poly (L-lactide-co-glycolide), poly(D,L-lactide-co-glycolide), poly (D,L-lactide-co-glycolide-D-glucose), poly (hydroxybutyric acid) and further derivatives, copolymers or mixtures thereof.

18. The method according to claim 5, in which the matrix material is a mixture of modified starch bearing octenyl succinate moieties and mannitol, admixed in a weight % ratio of from 80:20 to 50:50.

19. The method according to claim 6, in which the matrix material is a mixture of modified starch bearing octenyl succinate moieties and mannitol, admixed in a weight % ratio of from 80:20 to 50:50, and the organic liquid non-solvent is a polyethylene glycol (PEGs) having a molecular weight of from about 200 to about 700.
